(19) United States
(12) Patent Application Publication
(10) Pub. No.: US 2009/0156660 A1
(43) Pub. Date: Jun. 18, 2009

(54) PHARMACEUTICAL COMPOSITION CONTAINING TAXANE DERIVATIVE DESTINED FOR THE PREPARATION OF AN INFUSION SOLUTION, METHOD OF PREPARATION THEREOF AND USE THEREOF

(76) Inventors: Michal Svoboda, Praha (CZ); Xenia Svobodova, Praha (CZ); Martin Potucek, Ratnovce (SK); Vieroslav Kratky, Hlinik Nad Hronom (SK); Pavel Hanzlik, Praha (CZ)

Correspondence Address: NOTARO & MICHALOS P.C., 100 DUTCH HILL ROAD, SUITE 110, ORANGEBURG, NY, 10962-2100, US

(21) Appl. No.: 12/299,230

(22) PCT Filed: Mar. 26, 2007
(86) PCT No.: PCT/CZ2007/000018

§ 371 Date: Nov. 6, 2008

(30) Foreign Application Priority Data
May 3, 2006 (CZ) PV 2006-286
Dec. 20, 2006 (CZ) PV 2006-812

(51) Int. Cl. A61K 31/337

(52) U.S. Cl. 514/449

(57) ABSTRACT

A pharmaceutical composition containing a taxane derivative, destined for the preparation of an infusion solution for administration to patients, containing a concentrate consisting of a pharmaceutically effective amount of docetaxel, a suitable solvent, which is preferably ethanol, a surfactant, which is polysorbate 80 and a pharmaceutically effective amount of an appropriate buffer; and optionally a co-solvent, consisting of an aqueous solution of a pharmaceutically effective amount of polysorbate 80 and optionally suitable organic solvent and/or a pharmaceutically effective amount of an appropriate buffer. This composition shows an excellent chemical and physical stability. The invention includes also a method of preparation of the pharmaceutical composition and the use thereof.

### FIELD OF THE INVENTION

The invention relates to a novel pharmaceutical composition containing the taxane derivative docetaxel, destined for the preparation of an infusion solution, method of preparation thereof and use thereof. This composition shows an exceptional chemical and physical stability.

### BACKGROUND ART

Docetaxel is a chemotherapeutic possessing antitumor activity from the group of taxanes. It is prepared semi-synthetically, wherein the starting compound 10-deacetylbaccatin III is extracted from the needles of common yew (Taxus baccata), see EP 0253738 and EP 0336841 (Aventis Pharma). Alternative syntheses starting from different natural taxanes are also known, the starting compound being e.g. 9-dihydro-13-acetyl baccatin from the yew Taxus canadensis (EP 0639186, Abbott Lab.).

Docetaxel is in vivo effective against a large range of tumors. It is administered in particular in cases of breast carcinoma, but also in cases of ovarian and lung cancer. Later, its effectiveness in the treatment of hepatocellular carcinoma (EP1214061, Aventis Pharma) and other malignant cancers was described. The mechanism of action of docetaxel includes the inhibition of mitosis by influencing cellular microtubules, the decomposition of which into tubular proteins is prevented.

The taxane compounds (taxanes) generally show limited water solubility. Thus, the compositions, destined for the preparation of solutions suitable for the injection application, usually contain a surfactant and ethanol. Ethanol is the best biocompatible solvent for bringing docetaxel and other active compounds from the group of taxanes into solution.

Docetaxel was first described as a novel, biologically active compound in the French patent FR 2601675 (EP 0253 738). The patent claimed also a composition containing docetaxel for parenteral, particularly intravenous administration. For this composition, the use of auxiliary substances was proposed, said auxiliary substances being emulgators, dispersing agents or detergents (wetting agents), particularly propylene glycol, plant oils and injectable organic esters. In the example given in the above-mentioned patent, docetaxel was dissolved in the mixture of ethoxylated castor oil and ethanol in the ratio 1:1 (v/v) and the resulting mixture was subsequently diluted with saline solution in the ratio 1:9 (v/v). The composition was applied within 1 hour from the dilution.

With bringing taxanes, particularly taxol (paclitaxel) into solution generally deals an article published in Journal of the National Cancer Inst. 82(15), 1990, 1247-59. It describes a composition containing taxol, which is dissolved in the mixture consisting of 50% of dehydrated alcohol and 50% of ethoxylated castor oil (Cremophor EL). Similarly, in Chemical Abstract 106(22), 1987 (in the abstract No. 182 581c), an auxiliary solvent system is taught, consisting of polyethylene-polypropylene block copolymer (Pluronic L64), ethanol and polysorbate, which creates a stable composition with taxol that is applicable after dilution with water.

The use of surfactants (surface active agents, e.g. Cremofor) in the amount necessary for dissolving the therapeutically sufficient dose of the active substance induces undesired effects in patients, such as vasodilatation, breathlessness, decreased blood pressure and anaphylactic shock (see Journal of National Cancer Inst. 82(15), 1990, 1247-59 and Weiss et al., J. Clin. Oncol. 8, 1263-1268, 1990).

Moreover, the injectable pharmaceutical compositions in the surfactant, containing small amounts of ethanol, can be dissolved in the infusion solution solely under extremely vigorous stirring, which can be difficult to reach in the clinical workplace. According to EP 0671912 (Aventis Pharma), this disadvantage is overcome by the preparation of an intermediary solution, consisting of docetaxel in a surfactant and an aqueous solution with an additive, facilitating the dissolution of the intermediary solution in the aqueous infusion solution.

Stable pharmaceutical compositions containing docetaxel, destined for intravenous application, can be prepared using phospholipids (see EP 0758231, Aventis Pharma). Their advantage is almost the complete absence of pharmaceutically unsuitable organic solvents.

Another solution is a pharmaceutical composition, in which docetaxel is conjugated with a water-soluble polymer or a water-soluble chelating agent (EP 0932399, PG-TXL Company, L.P.).

Another way to bring docetaxel, which has a low water-solubility, into a solution for intravenous application is dissolution of docetaxel in plant oil, dilution with water and incorporation into liposomes or association with carriers such as cyclodextrins or polyethylene glycols (EP 0667771, Aventis Pharma).

A fat emulsion with phospholipids and an emulsifying protein that can be used also for incorporation of docetaxel into liposome emulsion is proposed in EP 1585504 (Azaya Therapeutics, Inc.). EP 1305006 (Pharmasol G.m.b.H.) describes emulsions of the oil-in-water type and water-in-oil type, not containing organic solvents, which can be used for formulating docetaxel as an injectable solution. Other compositions in the form of a fat emulsion are proposed in the patent application WO 2005065676 (Otsuka Pharma Factory Inc.). Another patent application of the same applicant describes a composition containing docetaxel in the solution of ethanol and polyethylene glycol that is further mixed with an infusion solution (JP 2005225818).

With the problem of bringing compounds having a low water-solubility (including docetaxel) into solution generally deals EP 1510206 (Novagali Pharma S.A.), proposing non-aqueous oil compositions spontaneously constituting nanoemulsions. Apart from the active agent, they contain vitamin E, an auxiliary solvent and a surfactant, optionally also a second biologically active compound. Another solution for the compounds having a low water-solubility is described in EP 1246608 (ImaRx Therapeutics, Inc.). The claimed injectable solution contains, apart from the active substance, polyvinyl pyrrolidone, a fatty acid and a surfactant.

EP 1560577 (Bristol-Myers Squibb Company) proposes docetaxel derivatives containing sulphur atom that are formulated into composition for the intravenous application using ethanol, polyoxyethylated castor oil and mixture of antioxidants.

The only so far registered medicament containing docetaxel, Taxotere®, is described in EP 0522936 (Aventis Pharma). The pharmaceutical composition consists of the solution of docetaxel in a surfactant (selected from polysorbates, polyoxyethylene glycol esters, polyethylene glycol esters and hydrogenated castor oil) with a small amount of ethanol. This stock solution is diluted for the application with saline solution or glucose solution. The resulting infusion solution shows the physical stability without the presence of ethanol in the range of several months. The same solution containing ethanol (the content of which is in the range of less than 0.01 ml/l to 0.05 ml/l) shows the stability of from 8 to 45 hours (see EP0522936, Aventis).

An analogical injectable solution is claimed in another patent of Aventis Pharma (EP 0671912), relating to taxane compounds including docetaxel. The pharmaceutical composition consists of docetaxel solution in a surfactant (selected from polysorbates or polyoxyethylene glycol esters and fatty acid glycerides) with a small amount of ethanol and diluting additive. The diluting additive consists of an organic compound having the molecular weight of less than 200 or a mineral salt and prevents the creation of gel phase after mixing the above-mentioned solution with the aqueous solution.

Another embodiment was proposed for the formulation of intravenously applied pharmaceutical compositions, containing taxane derivatives with sulphur atom. EP 1558241 (Bristol-Myers Squibb) describes a solution, consisting in the use of two containers, one of them containing the active compounds in a solvent in the presence of a buffer and the other container containing an auxiliary solvent in the presence of a buffer. The contents of both containers are to be mixed before the application of the composition.

Such compositions show an increased stability during shelf-life stocking and subsequent dilution with water, because the degradation of the taxane derivate by peroxide impurities from the polyoxyethylated castor oil, which serves as the auxiliary solvent, does not occur in these compositions.

The preferred composition of the pharmaceutical composition of the above-mentioned invention includes 0.001 to 20 mg/ml of the active component and 5% to 95% (v/v) of ethanol in aqueous solution of tartrate buffer in the first container and 1% to 95% (v/v) of polyoxyethylated castor oil in aqueous solution of tartrate buffer in the second container.

According to the above-mentioned patent, the solution of the pharmaceutical composition having acceptable stability and solubility can be easily prepared using 75% solution of ethanol in a buffer. However, for administration to patients it must be diluted with saline solution or glucose solution because of high content of ethanol. This procedure produces precipitation, which is superseded by the addition of the auxiliary solvent, preferably of polyoxyethylated castor oil. The stability of the injection solution, containing such an auxiliary solvent, is then much lower than in the absence thereof.

Now it was found that a concentrate containing docetaxel, dissolved in polysorbate 80 and ethanol with the addition of citrate buffer or any other physiologically acceptable buffer, shows surprisingly high chemical and physical stability. The determined stability in the range of several years was not described in similar pharmaceutical compositions until now. During parallel testing in a time range of from 6 to 12 days, the chemical stability of a concentrate containing docetaxel exceeded from tenfold to fifteen-fold the chemical stability of the commercially available composition Taxotere® (Aventis-Pharma). A high stability was confirmed also for a premix, obtained by the dilution of the concentrate with a co-solvent (the auxiliary solvent). The concentrate can be long-term stored in the range for commercial purposes, and even after long storing period, after the addition of the co-solvent and after a common dilution by the health care personnel it forms a stable infusion solution.

### DESCRIPTION OF THE INVENTION

Object of the present invention is a pharmaceutical composition, containing a concentrate consisting of:
a) a pharmaceutically effective amount of docetaxel,
b) a suitable solvent,
c) a surfactant (surface active agent), and
d) a pharmaceutically effective amount of a buffer;
   and optionally a co-solvent consisting of:
e) an aqueous solution of a pharmaceutically effective amount of a surfactant and optionally also
f) a suitable organic solvent and/or a pharmaceutically effective amount of a buffer.

It is an aspect of the present invention that the pharmaceutical composition of the invention contains a premix consisting of the mixture of the concentrate and the co-solvent.

It is another aspect of the present invention that the pharmaceutical composition of the invention contains the premix, which is adjusted to the form suitable for administration.

It is a further aspect of the present invention that the pharmaceutical composition of the invention contains the premix, which is adjusted to the form suitable for administration by the addition of an aqueous solution for infusions, selected from the group consisting of 0.9% aqueous solution of sodium chloride and 5% aqueous solution of glucose.

The suitable solvent for the pharmaceutical composition of the present invention is ethanol, applicable are also its mixtures with 2-propanol or 2-propanol alone. The suitable surfactant in accordance with the present invention is polysorbate 80.

It is an aspect of the pharmaceutical composition of the present invention that the suitable buffer is citrate buffer, tartrate buffer or lactate buffer or its combination with the mineral acid, preferably with hydrochloric acid or with its aqueous solution.

Object of the present invention is further the pharmaceutical composition containing the concentrate consisting of:
a) docetaxel or its hydrate in the amount, which corresponds to the amount of from 15 to 85 mg of anhydrous docetaxel in 1 ml of concentrate,
b) ethanol in the amount of from 3% to 57% (w/w),
c) polysorbate 80 in the amount of from 30% to 96% (w/w), and
d) from 1% to 25% (w/w) of the buffer having the concentration of from 47 to 250 mmol·l⁻¹ of citric acid monohydrate and 0.17 to 1.40 mmol·l⁻¹ of trisodium citrate dihydrate,
   and optionally the co-solvent, containing:
e) polysorbate 80 in the amount of from 5% to 35% (w/w), and optionally also
f) from 1% to 10% (w/w) of ethanol and/or the pharmaceutically effective amount of the buffer.

In a preferred embodiment the pharmaceutical composition of the present invention contains the concentrate consisting of:
a) docetaxel trihydrate in the amount of from 16.0 to 85.4 mg, which corresponds to the amount of from 15 to 85 mg of anhydrous docetaxel, in 1 ml of concentrate,
b) ethanol in the amount of from 3% to 57% (w/w),
c) polysorbate 80 in the amount of from 30% to 96% (w/w), and
d) from 1% to 25% (w/w) of the buffer having the concentration of from 47 to 250 mmol·l⁻¹ of citric acid monohydrate and 0.17 to 1.40 mmol·l⁻¹ of trisodium citrate dihydrate,
   and optionally the co-solvent, containing:
e) polysorbate 80 in the amount of from 5% to 35% (w/w), and optionally also
f) from 1% to 10% (w/w) of ethanol and/or the pharmaceutically effective amount of the buffer.

In another preferred embodiment, the pharmaceutical composition of the present invention contains the concentrate consisting of:
a) docetaxel or its hydrate in the amount, which corresponds to the amount of from 15 to 85 mg of anhydrous docetaxel in 1 ml of concentrate,
b) ethanol in the amount of from 3% to 57% (w/w),
c) polysorbate 80 in the amount of from 48% to 58% (w/w), and
d) from 1% to 25% (w/w) of the buffer having the concentration of from 47 to 250 mmol·l⁻¹ of citric acid monohydrate and 0.17 to 1.40 mmol·l⁻¹ of trisodium citrate dihydrate,
   and optionally the co-solvent, containing:
e) polysorbate 80 in the amount of from 5% to 35% (w/w), and optionally also
f) from 1% to 10% (w/w) of ethanol and/or the pharmaceutically effective amount of the buffer.

In further preferred embodiment, the pharmaceutical composition of the present invention contains the concentrate consisting of:
a) docetaxel trihydrate in the amount of from 16.0 to 85.4 mg, which corresponds to the amount of from 15 to 85 mg of anhydrous docetaxel, in 1 ml of concentrate,
b) ethanol in the amount of from 3% to 57% (w/w),
c) polysorbate 80 in the amount of from 48% to 58% (w/w), and
d) from 1% to 25% (w/w) of the buffer having the concentration of from 47 to 250 mmol·l⁻¹ of citric acid monohydrate and 0.17 to 1.40 mmol·l⁻¹ of trisodium citrate dihydrate,
   and optionally the co-solvent, containing:
e) polysorbate 80 in the amount of from 5% to 35% (w/w), and optionally also
f) from 1% to 10% (w/w) of ethanol and/or the pharmaceutically effective amount of the buffer.

Object of the present invention is further a pharmaceutical composition containing a taxane derivative, which contains a concentrate consisting of a pharmaceutically effective amount of docetaxel in the mixture of an organic solvent, a surfactant polysorbate 80 and a buffer.

It is an aspect of the present invention that the pharmaceutical composition of the invention contains the concentrate, which is adjusted to the form suitable for administration by the addition of an aqueous solution for infusions, selected from the group consisting of 0.9% aqueous solution of sodium chloride and 5% aqueous solution of glucose.

It is an aspect of the pharmaceutical composition of the present invention that the solvent is selected from the group consisting of ethanol, 2-propanol or their mixture, the surfactant is polysorbate 80 and the buffer is chosen from the group, consisting of citrate buffer, tartrate buffer or lactate buffer.

Object of the present invention is further the pharmaceutical composition containing the concentrate consisting of:
a) docetaxel or its hydrate in the amount, which corresponds to the amount of from 15 to 40 mg of anhydrous docetaxel in 1 ml of concentrate,
b) ethanol in the amount of from 3% to 57% (w/w),
c) polysorbate 80 the amount of from 30% to 96% (w/w), and
d) from 1% to 25% (w/w) of the buffer having the concentration of from 47 to 250 mmol·l⁻¹ of citric acid monohydrate and 0.17 to 1.40 mmol·l⁻¹ of trisodium citrate dihydrate.

In a preferred embodiment the pharmaceutical composition of the present invention contains the concentrate consisting of:
a) docetaxel trihydrate in the amount of from 16.0 to 42.4 mg, which corresponds to the amount of from 15 to 40 mg of anhydrous docetaxel, in 1 ml of concentrate,
b) ethanol in the amount of from 3% to 57% (w/w),
c) polysorbate 80 in the amount of from 30% to 96% (w/w), and
d) from 1% to 25% (w/w) of the buffer having the concentration of from 47 to 250 mmol·l⁻¹ of citric acid monohydrate and 0.17 to 1.40 mmol·l⁻¹ of trisodium citrate dihydrate.

In another preferred embodiment, the pharmaceutical composition of the present invention contains the concentrate consisting of:
a) docetaxel or its hydrate in the amount, which corresponds to the amount of from 15 to 40 mg of anhydrous docetaxel in 1 ml of concentrate,
b) ethanol in the amount of from 3% to 57% (w/w),
c) polysorbate 80 in the amount of from 60% to 70% (w/w), and
d) from 1% to 25% (w/w) of the buffer having the concentration of from 47 to 250 mmol·l⁻¹ of citric acid monohydrate and 0.17 to 1.40 mmol·l⁻¹ of trisodium citrate dihydrate.

In yet another preferred embodiment, the pharmaceutical composition of the present invention contains the concentrate consisting of:
a) docetaxel trihydrate in the amount of from 16.0 to 42.4 mg, which corresponds to the amount of from 15 to 40 mg of anhydrous docetaxel, in 1 ml of concentrate,
b) ethanol in the amount of from 3% to 57% (w/w),
c) polysorbate 80 in the amount of from 60% to 70% (w/w), and
d) from 1% to 25% (w/w) of the buffer having the concentration of from 47 to 250 mmol·⁻¹ of citric acid monohydrate and 0.17 to 1.40 mmol·l⁻¹ of trisodium citrate dihydrate.

Object of the present invention is further a method of preparation of the pharmaceutical composition of the invention, wherein the taxane derivative docetaxel dissolved in the solution consisting of the solvent, which is ethanol, 2-propanol or their mixture, polysorbate 80 and the buffer, is mixed with the solution consisting of an aqueous solution of the same solvent, polysorbate 80 and optionally also the buffer, for the preparation of the premix, which is subsequently adjusted to the form suitable for administration.

It is an aspect of the method of the present invention that the premix is adjusted to the form suitable for administration by the dilution in the ratio of 1 to 2 parts of the premix to 25 parts of the infusion with the sterile aqueous solution for infusions, selected from the group consisting of 0.9% aqueous solution of sodium chloride and 5% aqueous solution of glucose.

Object of the invention is further a method of preparation of the pharmaceutical composition of the present invention, wherein the taxane derivative docetaxel is dissolved in the solution consisting of the organic solvent which is ethanol, 2-propanol or their mixture, polysorbate 80 and the buffer, in order to obtain the concentrate, which is subsequently adjusted to the form suitable for administration.

It is a further aspect of the method of preparation of the pharmaceutical composition of the present invention that the concentrate is adjusted to the form suitable for administration by the dilution in the ratio of 1 part of the concentrate to 19-90 parts of the infusion with the sterile solution for infusions, selected from the group consisting of 0.9% aqueous solution of sodium chloride and 5% aqueous solution of glucose.

It is an aspect of the present invention that the pharmaceutical composition is adjusted to the form suitable for intravenous administration.

Object of the present invention is also the use of the pharmaceutical composition of the invention for the preparation of a medicament in the form suitable for intravenous administration destined for the treatment of various types of cancerous diseases and other diseases manifested by an abnormal proliferation of cells.

Object of the present invention is further a kit for inhibiting tumor growth, which comprises a concentrate, consisting of the taxane derivative docetaxel, a suitable organic solvent, a surfactant and a pharmaceutically effective amount of a buffer, and a co-solvent containing a pharmaceutically effective amount of the same surfactant, a suitable organic solvent and optionally a pharmaceutically effective amount of a buffer, wherein the concentrate and the co-solvent are destined for use in combination in order to prepare a premix, which is diluted with a sterile aqueous solution for infusions prior to administration to a patient.

The pharmaceutical composition of the invention is prepared in the way that the concentrate and the co-solvent are mixed prior to administration in order to prepare the premix (basic solution), which is subsequently diluted with saline solution (0.9% aqueous solution of sodium chloride) or with 5% aqueous solution of glucose just prior to intravenous application.

In a preferred embodiment, object of the invention is the pharmaceutical composition containing the concentrate of docetaxel in the mixture of the organic solvent, the surfactant and the buffer. For the preparation of the form suitable for administration, the concentrate is diluted with the prescribed amount of the sterile aqueous solution for infusions, i.e. 0.9% aqueous solution of sodium chloride or 5% aqueous solution of glucose just prior to administration to a patient.

The composition of the present invention provides an advantageous way for administrating the active substance docetaxel, while maintaining its solubility, substantially increasing the chemical stability of the compound during its shelf-life storage and the physical stability of the prepared concentrated solutions and the premix.

Docetaxel is a compound stabilizing cellular microtubules and as such, it can be used for the treatment of various types of cancerous diseases or other diseases, manifested by an abnormal cell proliferation. The composition of the invention is particularly suitable for use for the preparation of a medicament destined for the treatment of patients suffering from cancerous disease or other hyperproliferative cell disease. The term “cancerous disease” as used herein includes not only the carcinomas of soft and hard tissues and blood carcinomas, it also extends to skin, tissue, organ, bone, cartilage, blood and vessel diseases. The term “cancerous disease” further includes primary and metastasizing types of cancerous proliferation.

The composition of the invention is preferably provided in the form of dosage units in sealed vials, preferably in glass vials.

The organic solvent used in the composition of the present invention is preferably ethanol, which is the best biocompatible solvent for bringing docetaxel and also other active substances from the group of taxanes into solution. Besides ethanol also 2-propanol or their mixture can be used.

The concentration of docetaxel in the claimed composition is preferably 40 mg in one ml of the concentrate in the formulation utilizing the co-solvent. This concentration enables the preparation of the premix containing 10 mg of docetaxel in 1 ml. The premix is then in the amount needed (usually in the ratio of 1 to 2 parts of the premix to 25 parts of the infusion solution) diluted with the suitable aqueous solution for infusions and applied parenterally in the course of one hour.

The surfactant used in the composition of the invention is polysorbate 80, a non-ionic surface active, emulsifying substance. It is a sorbitol derivative, chemically it is polyoxyethylene (20) sorbitan monooleate.

This surfactant was selected for the reasons of good dissolving capacity for docetaxel and the ability to maintain this compound in the aqueous solution for a long period without precipitating. The pharmaceutical compositions containing polysorbate 80 appear to be more advantageous for parenteral administration than the compositions containing ethoxylated castor oil with regard to the different pharmacokinetical behaviour of polysorbate 80. This compound is eliminated much faster from the plasma of patients by hydrolysis in the presence of carboxylesterase (see Clin. Pharmakokinetics 2003, 42(7), 665-685).

In the concentrate the composition of the present invention contains polysorbate 80 in the amount of 30 to 96% w/w, preferably of 48 to 58% w/w with respect to the weight of the concentrate. The amount contained in the co-solvent is 5 to 35% w/w, preferably 16 to 20% w/w with respect to the weight of the co-solvent. Thus, one milligram of docetaxel in the form suitable for administration (in the infusion solution) corresponds to 11 to 50 mg of polysorbate 80, preferably 24.0 to 30.0 mg. After the preparation of the premix and the dilution thereof, the highest concentration of polysorbate in the form suitable for administration is 3.5% w/w; usually it ranges from 0.2 to 1.6% w/w with respect to the weight of the resulting infusion solution as the form suitable for administration.

In a preferred embodiment the composition of the invention without the co-solvent contains in the concentrate polysorbate 80 in the amount of 30 to 96% w/w, preferably 60 to 70% w/w with respect to the weight of the composition. Thus, one milligram of docetaxel in the form suitable for administration corresponds to 12 to 39 mg of polysorbate 80, preferably 20 to 30 mg. After the preparation of the premix and the dilution thereof, the highest concentration of polysorbate 80 in the form suitable for administration is 2.8% w/w; usually it ranges from 0.2 to 1.9% w/w with respect to the weight of the resulting infusion solution as the form suitable for administration.

It was found that in order to achieve the optimal stability of the composition, said composition must contain a pharmaceutically acceptable buffer with excess of the acidic component. The preferred buffer is citrate buffer prepared in excess of citric acid. The buffer preferably comprises 990 mg to 5300 mg of citric acid monohydrate and 5 mg to 40 mg of trisodium citrate dihydrate in 100 ml of the solution. Tartrate buffer and lactate buffer belong among other pharmaceutically acceptable buffers.

The chemical stability of the concentrate of the composition of the present invention depends substantially on the pH value and the portion of an aqueous constituent as well. The required value of pH of the concentrate can be achieved by addition of an appropriate buffer or its combination with hydrochloric acid or its aqueous solution. The precise amount of this addition can be calculated on the basis of the known content of free acids and bases in polysorbate 80 and the desired final content of water.

The preferred composition of the present invention contains the concentrate consisting of:
a) docetaxel or its hydrate in the amount which corresponds to the amount of from 15 to 85 mg of anhydrous docetaxel in 1 ml of concentrate,
b) ethanol in the amount of from 3% to 57% (w/w),
c) polysorbate 80 in the amount of from 30% to 96% (w/w), and
d) from 1% to 25% (w/w) of the buffer having the concentration of from 47 to 250 mmol·l⁻¹ of citric acid monohydrate and 0.17 to 1.40 mmol·l⁻¹ of trisodium citrate dihydrate,
   and optionally the co-solvent, containing:
e) polysorbate 80 in the amount of from 5% to 35% (w/w), and optionally also
f) from 1% to 10% (w/w) of ethanol and/or the pharmaceutically effective amount of the buffer.

In a preferred embodiment, the composition of the present invention contains only the concentrate consisting of:
a) docetaxel or its hydrate in such amount which corresponds to the amount of from 15 to 40 mg of anhydrous docetaxel in 1 ml of concentrate,
b) ethanol in the amount of from 3% to 57% (w/w),
c) polysorbate 80 in the amount of from 30% to 96% (w/w), and
d) from 1% to 25% (w/w) of the buffer of the concentration of from 47 to 250 mmol·l⁻¹ of citric acid monohydrate and 0.17 to 1.40 mmol·l⁻¹ of trisodium citrate dihydrate.

The particularly preferred compositions of the pharmaceutical composition of the present invention are shown in detail in Table 1.

The invention is hereinafter illustrated by the following examples, which should not be construed as further limiting.

### FIGURES

FIGS. 1A to 1D represent charts showing the course of the contents of impurities formed by the decomposition of docetaxel in a sample of the concentrate of the pharmaceutical composition having the composition according to Example 1 in time. At the y-axis the total amount of impurities (symbol ▪) and the amount of two selected impurities showing the fastest increase (symbol for 10-oxo-docetaxel as the impurity 1 and ▴ for 7-epidocetaxel as the impurity 2) is outlined as weight % with respect to the weight of the sample. At the x-axis the time is outlined as hours.

FIG. 1A represents the above-captioned chart at the temperature 25° C.;

FIG. 1B represents the above-captioned chart at the temperature 40° C.;

FIG. 1C represents the above-captioned chart at the temperature 55° C.; and

FIG. 1D represents the above-captioned chart at the temperature 70° C.

FIGS. 2A to 2D represent charts showing the course of the contents of impurities formed by the decomposition of docetaxel in a sample of the concentrate of the pharmaceutical composition having the composition according to Example 2 in time. At the y-axis the total amount of impurities (symbol ▪) and the amount of two selected impurities showing the fastest increase (symbol for 10-oxo-docetaxel as the impurity 1 and ▴ for 7-epidocetaxel as the impurity 2) is outlined as weight % with respect to the weight of the sample. At the x-axis the time is outlined as hours.

FIG. 2A represents the above-captioned chart at the temperature 25° C.;

FIG. 2B represents the above-captioned chart at the temperature 40° C.;

FIG. 2C represents the above-captioned chart at the temperature 55° C.; and

FIG. 2D represents the above-captioned chart at the temperature 70° C.

FIG. 3 shows the comparison of the chemical stability of the composition of the present invention having the composition according to Table IV and commercially available medicament Taxotere®, where the content of impurities formed by the decomposition of docetaxel is measured depending on time. At the y-axis, the total amount of impurities is outlined as weight % with respect to the weight of the sample. At the x-axis, the time is outlined as hours.

### EXAMPLES

### Chemical Stability of the Composition

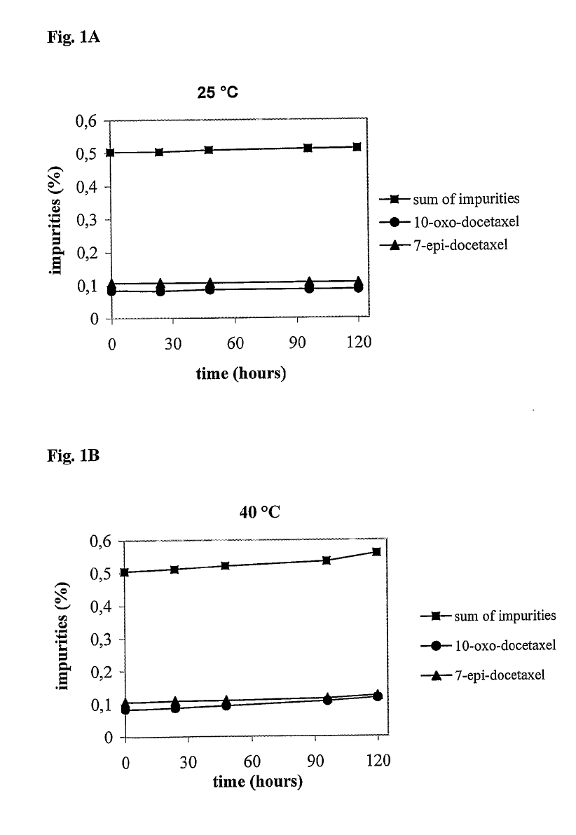

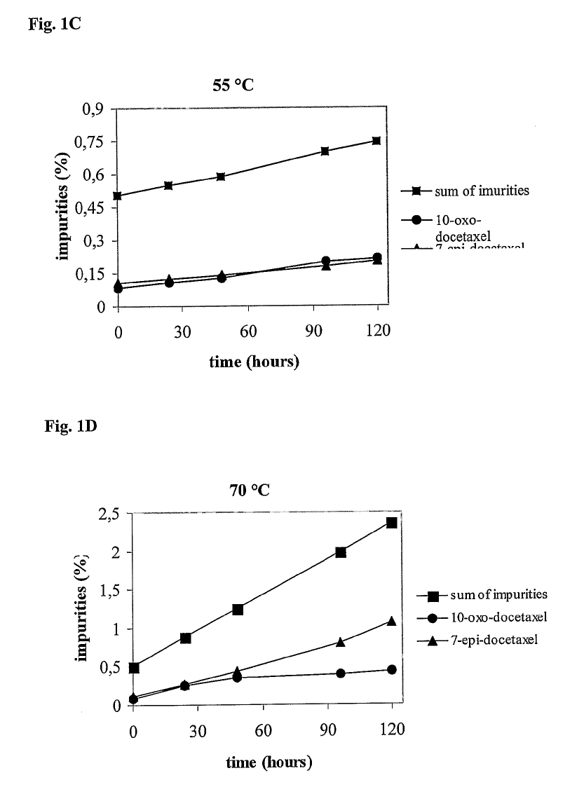

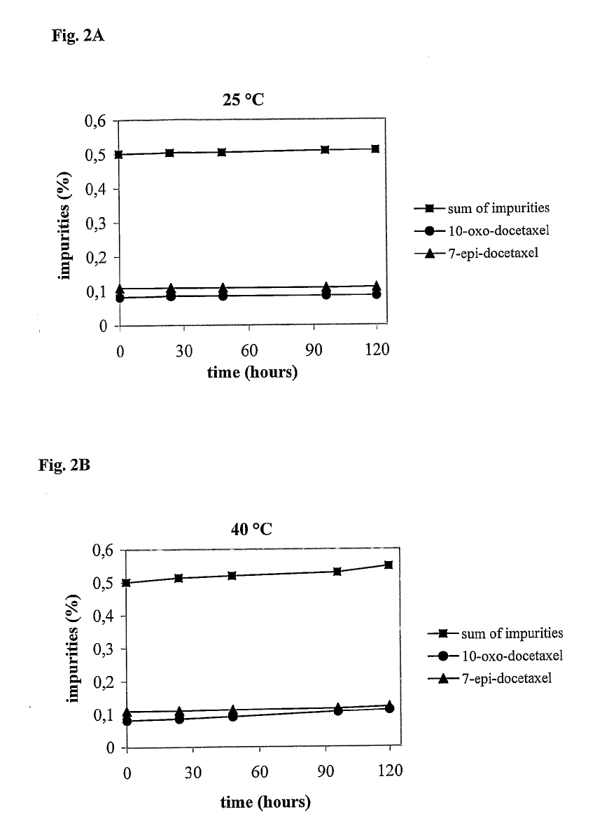

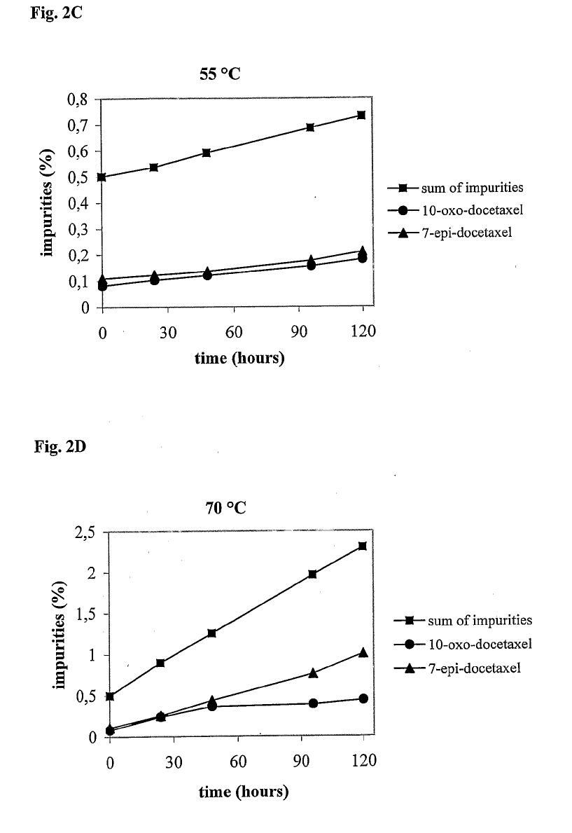

The chemical stability of the composition of the present invention was tested in the concentrate. The percentages of the sum of impurities and of two selected impurities showing the fastest increase, formed by the decomposition of docetaxel, in dependence on time were measured at various temperatures. The claimed composition, the compositions of which are shown in Table I, was exposed to the temperatures 25, 40, 55 and 70° C. for up to 120 hours. The percentage increases of the sum of impurities and separately of two major impurities in the samples were determined by HPLC analysis, see FIGS. 1A to 1D, 2A to 2D and tables II and III.

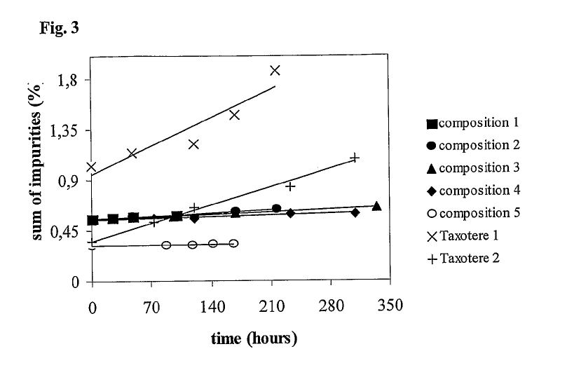

The chemical stability of the claimed composition was also compared with the chemical stability of two independent, in parallel tested batches of the medicament Taxotere®, commercially available from Aventis-Pharma (see FIG. 3). The composition of the present invention, the compositions of which are shown in Table IV, was from tenfold to fifteen-fold more chemically stable than Taxotere® in the time range from 0 up to 310 hours at the temperature 40° C. The time dependent percentual increases of the sum of 10-oxo-docetaxel and 7-epidocetaxel, formed by the decomposition of docetaxel, are shown in Table V.

The increases were assessed also by the methods of mathematical regression analysis enabling the extrapolation of the time period for achieving a given limit concentration of the impurities. The tested compositions of the pharmaceutical composition of the invention show extremely low impurities increases in dependence on time; the mathematical extrapolation to the laboratory temperature confirmed the high stability of the composition, exceeding three years.

This stability is attributed to the new composition of the pharmaceutical composition, which comprises, besides the taxane derivative, also ethanol, polysorbate 80 and the aqueous solution of buffer. The analogous results, obtained using the alternative solvents, are not shown here.

The physical stability of the premix of the present invention, defined by the formation of a turbidity (precipitation of docetaxel), was at least 2 hours from the preparation of premix (data not shown).

TABLE I
The compositions of the pharmaceutical composition
according to Examples 1 and 2

| | Composition | | | |
|---|---|---|---|---|
| | according to Example 1 | | according to Example 2 | |
| | con- | co- | con- | co- |
| | centrate: | solvent: | centrate: | solvent: |
| Component | mg/ml | g/100 ml | mg/ml | g/100 ml |
| Docetaxel | 42.68¹ | — | 42.68 | — |
| Ethanol | 300.0 | 2.71 | 380.0 | — |
| Polysorbate | 570.0 | 15.67 | 465.0 | 19.17 |
| 80 | | | | |
| Buffer² | 80.0 | — | 100.0 | — |
| Purified water | — | q.s. to 100 ml | — | q.s. to 100 ml |

Remarks:
¹Corresponds to 40 mg of anhydrous docetaxel.
²Composition of the buffer:
1. Citric acid monohydrate 1300 mg
2. Trisodium citrate dihydrate 40 mg
3. Purified water q.s. to 100 ml

### Example 1

### Preparation of the Concentrate

852.8 mg of docetaxel and 6.0 g of ethanol are weighed into a flask and the mixture is shaken until the substance is completely dissolved. 11.4 g of polysorbate 80 is added to the solution and the mixture is thoroughly stirred again.

An aqueous solution of the buffer is subsequently prepared, which consists of citric acid monohydrate and trisodium citrate dihydrate having the concentration of 62 mmol·l⁻¹ of the acid and of 1.4 mmol·l⁻¹ of the salt. 1.6 g of this buffer is added into the flask containing the solution of docetaxel in ethanol and polysorbate 80 and the mixture is stirred until a homogenous solution is formed. The thus prepared concentrate has the concentration of docetaxel 40 mg in one millilitre of the solution.

From the thus prepared concentrate a sample is taken, in which the impurities profile is determined by means of HPLC analysis, 10 ml is taken for the preparation of the premix and the residue is divided into four well-sealed containers, stored at the temperatures 25, 40, 55 and 70° C. In the intervals 24, 48, 96 and 120 hours, a sample is taken from each container and the percentage of impurities in this sample is determined. The results for the sum of impurities and two selected impurities are shown in Table II and FIGS. 1A to 1D.

TABLE II
The chemical stability of the concentrate of the composition according to
Example 1, expressed as weight % with respect to the weight of the sample.

| | Time | | | | | | | | | | | | | | | | |
|---|---|---|---|---|---|---|---|---|---|---|---|---|---|---|---|---|---|
| | | 24 hours | | | | 48 hours | | | | 96 hours | | | | 120 hours | | | |
| | 0 | ° C. | | | | | | | | | | | | | | | |
| Impurity | hours | 25 | 40 | 55 | 70 | 25 | 40 | 55 | 70 | 25 | 40 | 55 | 70 | 25 | 40 | 55 | 70 |
| 10-oxo- | 0.084 | 0.082 | 0.088 | 0.108 | 0.253 | 0.086 | 0.094 | 0.128 | 0.352 | 0.086 | 0.108 | 0.201 | 0.389 | 0.088 | 0.118 | 0.214 | 0.436 |
| docetaxel | | | | | | | | | | | | | | | | | |
| 7-epidocetaxel | 0.106 | 0.106 | 0.109 | 0.124 | 0.269 | 0.107 | 0.110 | 0.141 | 0.431 | 0.107 | 0.116 | 0.180 | 0.803 | 0.08 | 0.124 | 0.204 | 1.061 |
| Sum of | 0.504 | 0.504 | 0.511 | 0.549 | 0.886 | 0.509 | 0.521 | 0.587 | 1.244 | 0.512 | 0.535 | 0.699 | 1.975 | 0.514 | 0.559 | 0.744 | 2.366 |
| impurities | | | | | | | | | | | | | | | | | |

### Preparation of the Co-Solvent

Further, the solution of the co-solvent is prepared: 2.71 g of ethanol, 15.67 g of polysorbate 80 is weighed into a clean flask and the volume of the mixture is adjusted to 100 ml with purified water. The mixture is subsequently stirred until a homogeneous solution is formed.

### Preparation of the Premix

The premix is prepared by thoroughly admixing 10 ml of the concentrate with 30 ml of the co-solvent. The thus prepared premix has the concentration of 10 mg of docetaxel in 1 ml of the solution.

### Example 2

The preparation process is the same as in Example 1. For the preparation of the concentrate 7.6 g of ethanol, 9.3 g of polysorbate 80 and 2.0 g of the buffer having the same composition as in Example 1 are used. The co-solvent consists of 19.17% (w/w) solution of polysorbate 80.

The results relating to the chemical stability, expressed as the percentage of the sum of impurities and two selected impurities are shown in Table III and FIGS. 2A to 2D.

TABLE III
The chemical stability of the concentrate of the composition according to
Example 2, expressed as weight % with respect to the weight of the sample.

| | Time | | | | | | | | | | | | | | | | |
|---|---|---|---|---|---|---|---|---|---|---|---|---|---|---|---|---|---|
| | | 24 hours | | | | 48 hours | | | | 96 hours | | | | 120 hours | | | |
| | 0 | ° C. | | | | | | | | | | | | | | | |
| Impurity | hours | 25 | 40 | 55 | 70 | 25 | 40 | 55 | 70 | 25 | 40 | 55 | 70 | 25 | 40 | 55 | 70 |
| 10-oxo- | 0.082 | 0.085 | 0.086 | 0.103 | 0.242 | 0.085 | 0.091 | 0.120 | 0.364 | 0.086 | 0.106 | 0.155 | 0.390 | 0.086 | 0.111 | 0.181 | 0.441 |
| docetaxel | | | | | | | | | | | | | | | | | |
| 7-epidocetaxel | 0.109 | 0.108 | 0.110 | 0.122 | 0.255 | 0.109 | 0.112 | 0.136 | 0.437 | 0.109 | 0.116 | 0.176 | 0.759 | 0.111 | 0.121 | 0.210 | 1.006 |
| Sum of | 0.501 | 0.504 | 0.514 | 0.536 | 0.900 | 0.505 | 0.520 | 0.590 | 1.254 | 0.510 | 0.529 | 0.685 | 1.965 | 0.511 | 0.548 | 0.730 | 2.303 |
| impurities | | | | | | | | | | | | | | | | | |

The results relating to the comparison of the chemical stability of the composition according to the invention and of the commercially available medicament Taxotere® are summarized in Table V and in FIG. 3. The evaluation was carried out by the same procedure as described in example 1; the used compositions of the tested composition are shown in Table IV.

TABLE IV
The compositions of the pharmaceutical composition
for the chemical stability evaluation in comparison
with a commercially available preparative.

| | Content of component [mg/ml of concentrate] | | | | |
|---|---|---|---|---|---|
| Component | Compos. 1 | Compos. 2 | Compos. 3 | Compos. 4 | Compos. 5 |
| Docetaxel¹ | 42.67 | 42.86 | 43.05 | 42.76 | 40.02 |
| Ethanol | 300.18 | 300.27 | 380.14 | 299.98 | 300.12 |
| Polysorbate | 557.73 | 556.73 | 477.06 | 561.14 | 557.23 |
| 80 | | | | | |
| Buffer² | 79.63 | 80.75 | 78.73 | 80.90 | 82.24 |
| In sum | 980.21 | 980.61 | 978.98 | 984.78 | 979.61 |

Remarks:
¹Corresponds to 40 mg of anhydrous docetaxel.
²Composition of the buffer:
1. Citric acid monohydrate 1300 mg
2. Trisodium citrate dihydrate 40 mg
3. Purified water q.s. to 100 ml

TABLE V
Comparison of the chemical stability of the compositions
according to Table IV and the medicament Taxotere ®,
expressed as the weight % of the sum of impurities with
regards to the weight of the sample

| composition | | composition | | composition | | composition | |
|---|---|---|---|---|---|---|---|
| 1 | | 2 | | 3 | | 4 | |
| | sum of | | sum of | | sum of | | sum of |
| time | impuri- | time | impuri- | time | impuri- | time | impuri- |
| (h) | ties (%) | (h) | ties (%) | (h) | ties (%) | (h) | ties (%) |
| 0 | 0.55 | 0 | 0.54 | 0 | 0.55 | 0 | 0.55 |
| 24 | 0.56 | 48 | 0.57 | 96 | 0.57 | 73 | 0.55 |
| 48 | 0.57 | 120 | 0.59 | 168 | 0.6 | 120 | 0.55 |
| 98 | 0.59 | 168 | 0.61 | 335 | 0.65 | 232 | 0.6 |
| — | — | 216 | 0.63 | — | — | 310 | 0.6 |

| | | |
|---|---|---|
| composition | | |
| 5* | Taxotere 1 | Taxotere 2 |

| | sum of | | sum of | | sum of |
|---|---|---|---|---|---|
| time | impuri- | time | impuri- | time | impuri- |
| (h) | ties (%) | (h) | ties (%) | (h) | ties (%) |
| 0 | 0.36 | 0 | 1.02 | 0 | 0.35 |
| 87 | 0.36 | 48 | 1.13 | 73 | 0.52 |
| 118 | 0.37 | 120 | 1.22 | 120 | 0.65 |
| 142 | 0.37 | 168 | 1.48 | 232 | 0.83 |
| 166 | 0.38 | 216 | 1.86 | 310 | 1.08 |

*composition containing docetaxel anhydride

1: A pharmaceutical composition containing a taxane derivative, characterized in that it contains a concentrate consisting of:
a) a pharmaceutically effective amount of docetaxel,
b) a suitable solvent,
c) a surfactant and
d) a pharmaceutically effective amount of a buffer;
and optionally a co-solvent consisting of:
e) an aqueous solution of a pharmaceutically effective amount of a surfactant and optionally also
f) a suitable organic solvent and/or a pharmaceutically effective amount of a buffer.

2: The pharmaceutical composition according to claim 1, characterized in that it contains a premix consisting of the mixture of the concentrate and the co-solvent.

3: The pharmaceutical composition according to claim 2, characterized in that it contains the premix, which is adjusted to the form suitable for administration.

4: The pharmaceutical composition according to claim 3, characterized in that it contains the premix, which is adjusted to the form suitable for administration by the addition of an aqueous solution for infusions, selected from the group consisting of 0.9% aqueous solution of sodium chloride and 5% aqueous solution of glucose.

5: The pharmaceutical composition according to claim 1, characterized in that the suitable solvent is selected from the group, consisting of ethanol, 2-propanol and their mixture.

6: The pharmaceutical composition according to claim 5, characterized in that the suitable solvent is ethanol.

7: The pharmaceutical composition according to claim 1, characterized in that the suitable surfactant is polysorbate 80.

8: The pharmaceutical composition according to claim 1, characterized in that the suitable buffer is a pharmaceutically acceptable buffer or its combination with a mineral acid.

9: The pharmaceutical composition according to claim 8, characterized in that the suitable buffer is selected from the group, consisting of a citrate buffer, tartrate buffer, lactate buffer and a combination of one of said buffers with hydrochloric acid or its aqueous solution.

10: The pharmaceutical composition according to claim 9, characterized in that the particularly suitable buffer is citrate buffer or its combination with hydrochloric acid or with an aqueous solution of hydrochloric acid.

11: The pharmaceutical composition according to claim 1, characterized in that it contains the concentrate consisting of:
a) docetaxel or its hydrate in the amount, which corresponds to the amount of from 15 to 85 mg of anhydrous docetaxel in 1 ml of concentrate,
b) ethanol in the amount of from 3% to 57% (w/w),
c) polysorbate 80 in the amount of from 30% to 96% (w/w), and
d) from 1% to 25% (w/w) of the buffer having the concentration of from 47 to 250 mmol·l⁻¹ of citric acid monohydrate and 0.17 to 1.40 mmol·l⁻¹ of trisodium citrate dihydrate,
and optionally the co-solvent, containing:
e) polysorbate 80 in the amount of from 5% to 35% (w/w), and optionally also
f) from 1% to 10% (w/w) of ethanol and/or the pharmaceutically effective amount of the buffer.

12: The pharmaceutical composition according to claim 11, characterized in that it contains the concentrate consisting of:
a) docetaxel trihydrate in the amount of from 16.0 to 85.4 mg, which corresponds to the amount of from 15 to 85 mg of anhydrous docetaxel, in 1 ml of concentrate,
b) ethanol in the amount of from 3% to 57% (w/w),
c) polysorbate 80 in the amount of from 30% to 96% (w/w), and
d) from 1% to 25% (w/w) of the buffer having the concentration of from 47 to 250 mmol·l⁻¹ of citric acid monohydrate and 0.17 to 1.40 mmol·l⁻¹ of trisodium citrate dihydrate,
and optionally the co-solvent, containing:
e) polysorbate 80 in the amount of from 5% to 35% (w/w), and optionally also
f) from 1% to 10% (w/w) of ethanol and/or the pharmaceutically effective amount of the buffer.

13: The pharmaceutical composition according to claim 11, characterized in that it contains the concentrate consisting of:
a) docetaxel or its hydrate in the amount which corresponds to the amount of from 15 to 85 mg of anhydrous docetaxel in 1 ml of concentrate,
b) ethanol in the amount of from 3% to 57% (w/w),
c) polysorbate 80 in the amount of from 48% to 58% (w/w), and
d) from 1% to 25% (w/w) of the buffer having the concentration of from 47 to 250 mmol·l⁻¹ of citric acid monohydrate and 0.17 to 1.40 mmol·l⁻¹ of trisodium citrate dihydrate,
and optionally the co-solvent, containing:
e) polysorbate 80 in the amount of from 5% to 35% (w/w), and optionally also
f) from 1% to 10% (w/w) of ethanol and/or the pharmaceutically effective amount of the buffer.

14: The pharmaceutical composition according to claim 12, characterized in that it contains the concentrate consisting of:
a) docetaxel trihydrate in the amount of from 16.0 to 85.4 mg, which corresponds to the amount of from 15 to 85 mg of anhydrous docetaxel, in 1 ml of concentrate,
b) ethanol in the amount of from 3% to 57% (w/w),
c) polysorbate 80 in the amount of from 48% to 58% (w/w), and
d) from 1% to 25% (w/w) of the buffer having the concentration of from 47 to 250 mmol·l⁻¹ of citric acid monohydrate and 0.17 to 1.40 mmol·l⁻¹ of trisodium citrate dihydrate,
and optionally the co-solvent, containing:
e) polysorbate 80 in the amount of from 5% to 35% (w/w), and optionally also
f) from 1% to 10% (w/w) of ethanol and/or the pharmaceutically effective amount of the buffer.

15: A pharmaceutical composition containing a taxane derivative, characterized in that it contains a concentrate consisting of a pharmaceutically effective amount of docetaxel in the mixture of a solvent, a surfactant and a buffer.

16: The pharmaceutical composition according to claim 15, characterized in that it contains the concentrate adjusted to the form suitable for administration.

17: The pharmaceutical composition according to claim 16, characterized in that the concentrate is adjusted to the form suitable for administration by the addition of an aqueous solution for infusions, selected from the group consisting of 0.9% aqueous solution of sodium chloride and 5% aqueous solution of glucose.

18: The pharmaceutical composition according to claim 15, characterized in that the suitable solvent is selected from the group, consisting of ethanol, 2-propanol and their mixture.

19: The pharmaceutical composition according to claim 18, characterized in that the suitable solvent is ethanol.

20: The pharmaceutical composition according to claim 15, characterized in that the surfactant is polysorbate 80.

21: The pharmaceutical composition according to claim 15, characterized in that the suitable buffer is a pharmaceutically acceptable buffer or its combination with a mineral acid.

22: The pharmaceutical composition according to claim 21, characterized in that the suitable buffer is selected from the group, consisting of a citrate buffer, tartrate buffer, lactate buffer and a combination of one of said buffers with hydrochloric acid or its aqueous solution.

23: The pharmaceutical composition according to claim 22, characterized in that the particularly suitable buffer is citrate buffer or its combination with hydrochloric acid or with an aqueous solution of hydrochloric acid.

24: The pharmaceutical composition according to claim 15, characterized in that it contains the concentrate consisting of:
a) docetaxel or its hydrate in the amount which corresponds to the amount of from 15 to 40 mg of anhydrous docetaxel in 1 ml of concentrate,
b) ethanol in the amount of from 3% to 57% (w/w),
c) polysorbate 80 in the amount of from 30% to 96% (w/w), and
d) from 1% to 25% (w/w) of the buffer having the concentration of from 47 to 250 mmol·l⁻¹ of citric acid monohydrate and 0.17 to 1.40 mmol·l⁻¹ of trisodium citrate dihydrate.

25: The pharmaceutical composition according to claim 24, characterized in that it contains the concentrate consisting of:
a) docetaxel trihydrate in the amount of from 16.0 to 42.4 mg, which corresponds to the amount of from 15 to 40 mg of anhydrous docetaxel, in 1 ml of concentrate,
b) ethanol in the amount of from 3% to 57% (w/w),
c) polysorbate 80 in the amount of from 30% to 96% (w/w), and
d) from 1% to 25% (w/w) of the buffer having the concentration of from 47 to 250 mmol·l⁻¹ of citric acid monohydrate and 0.17 to 1.40 mmol·l⁻¹ of trisodium citrate dihydrate.

26: The pharmaceutical composition according to claim 15, characterized in that it contains the concentrate consisting of:
a) docetaxel or its hydrate in the amount which corresponds to the amount of from 15 to 40 mg of anhydrous docetaxel in 1 ml of concentrate,
b) ethanol in the amount of from 3% to 57% (w/w),
c) polysorbate 80 in the amount of from 60% to 70% (w/w), and
d) from 1% to 25% (w/w) of the buffer having the concentration of from 47 to 250 mmol·l⁻¹ of citric acid monohydrate and 0.17 to 1.40 mmol·l⁻¹ of trisodium citrate dihydrate.

27: The pharmaceutical composition according to claim 26, characterized in that it contains the concentrate consisting of:
a) docetaxel trihydrate in the amount of from 16.0 to 42.4 mg, which corresponds to the amount of from 15 to 40 mg of anhydrous docetaxel, in 1 ml of concentrate,
b) ethanol in the amount of from 3% to 57% (w/w),
c) polysorbate 80 in the amount of from 60% to 70% (w/w), and
d) from 1% to 25% (w/w) of the buffer having the concentration of from 47 to 250 mmol·l⁻¹ of citric acid monohydrate and 0.17 to 1.40 mmol·l⁻¹ of trisodium citrate dihydrate.

28: Method of preparation of the pharmaceutical composition of claim 1, characterized in that the taxane derivative docetaxel in the solution consisting of a solvent, surfactant polysorbate 80 and a buffer is mixed with the solution consisting of an aqueous solution of a solvent, polysorbate 80, and optionally a buffer for the preparation of the premix, which is subsequently adjusted to the form suitable for administration.

29: The method of preparation of the pharmaceutical composition according to claim 28, characterized in that the suitable solvent is selected from the group, consisting of ethanol, 2-propanol and their mixture.

30: The method of preparation of the pharmaceutical composition according to claim 29, characterized in that the particularly suitable solvent is ethanol.

31: The method according to claim 28, characterized in that the premix is adjusted to the form suitable for administration by the dilution in the ratio of 1 to 2 parts of the premix to 25 parts of infusion with an aqueous solution for infusions, selected from the group consisting of 0.9% aqueous solution of sodium chloride and 5% aqueous solution of glucose.

32: Method of preparation of the pharmaceutical composition of claim 15, characterized in that the taxane derivative docetaxel is dissolved in the solution consisting of solvent, polysorbate 80, and a buffer in order to obtain the concentrate, which is subsequently adjusted to the form suitable for administration.

33: The method of preparation of the pharmaceutical composition according to claim 32, characterized in that the suitable solvent is selected from the group, consisting of ethanol, 2-propanol and their mixture.

34: The method of preparation of the pharmaceutical composition according to claim 33, characterized in that the particularly suitable solvent is ethanol.

35: The method of preparation of the pharmaceutical composition according to claim 32, characterized in that the concentrate is adjusted to the form suitable for administration by the dilution in the ratio of 1 part of the concentrate into 19-90 parts of the infusion with an aqueous solution for infusions, selected from the group consisting of 0.9% aqueous solution of sodium chloride and 5% aqueous solution of glucose.

36: The method of preparation of the pharmaceutical composition according to claim 28, characterized in that the composition is adjusted to the form suitable for intravenous administration.

37: The use of the pharmaceutical composition according to claim 1 for the preparation of a medicament in the form suitable for intravenous administration for the treatment of various types of cancerous diseases and other diseases manifested by an abnormal proliferation of cells.

38: A kit for inhibiting the tumor growth, characterized in that it comprises a concentrate, consisting of the taxane derivative docetaxel, a suitable solvent, a surfactant and a pharmaceutically effective amount of a buffer, and a co-solvent containing a pharmaceutically effective amount of a surfactant, a suitable solvent and optionally also a pharmaceutically effective amount of a buffer, wherein the concentrate and the co-solvent are destined for use in combination in order to prepare a premix, which is diluted with a sterile aqueous solution for infusions prior to administration to a patient.
